(19) United States
(12) Patent Application Publication
(10) Pub. No.: US 2015/0314295 A1
(43) Pub. Date: Nov. 5, 2015

(54) STERILE FLUID HANDLING DEVICE

(71) Applicant: MR1 Holdings, LLC, Aiken, SC (US)

(72) Inventors: Adrian LANGLEY, Lexington, SC (US); Patrick Martin O'MALLEY, Chapin, SC (US); Michael David HORNING, Irmo, SC (US)

(21) Appl. No.: 14/704,522

(22) Filed: May 5, 2015

Related U.S. Application Data
(60) Provisional application No. 61/988,730, filed on May 5, 2014.

(51) Int. Cl. B01L 9/06; G01N 1/18; A61B 10/00

(52) U.S. Cl. CPC B01L 9/06; A61B 10/0096; G01N 1/18; B01L 2200/087; B01L 2200/141; B01L 2300/021; B01L 2300/042; B01L 2300/0609

(57) ABSTRACT

A sterile fluid handling device, comprising: a plate structure; one of a plurality of holes, wells, or recesses disposed in or on the plate structure, wherein each of the holes, wells, or recesses is sized and configured to selectively receive and retain a corresponding fluid collection tube; and a gripping portion by which the plate structure is grasped by a user and moved while the fluid collection tubes are retained in the holes, wells, or recesses. The device further comprising an elongate recess manufactured into the plate structure, wherein the elongate recess is sized and configured to receive an inner stylet of a fluid collection needle. The holes, wells, or recesses are arranged in a substantially linear pattern spanning a dimension of the plate structure. Alternatively, the holes, wells, or recesses are arranged in a substantially curved pattern spanning a dimension of the plate structure.

### CROSS-REFERENCE TO RELATED APPLICATION

The present patent application/patent claims the benefit of priority of co-pending U.S. Provisional Patent Application No. 61/988,730, filed on May 5, 2014, and entitled “STERILE FLUID HANDLING DEVICE,” the contents of which are incorporated in full by reference herein.

### FIELD OF THE INVENTION

The present invention relates generally to a sterile fluid handling device and method. More specifically, the present invention relates to a sterile fluid handling device and method for use in spinal lumbar puncture (LP) procedures and the like.

### BACKGROUND OF THE INVENTION

During a LP procedure, which is used to diagnose various illnesses, a patient sits or lies in a fetal, upright, or prone position on a hospital table while a physician inserts a relatively large bore needle with an inner stylet between two lumbar vertebrae in the patient's lower back. If performed properly, spinal fluid will immediately begin to drip from the needle bore once the inner stylet is removed. The spinal fluid will continue to drip until the stylet is placed back in the needle and the needle is removed from the spine.

In a standard LP procedure, the physician holds a series of test tubes under the dripping spinal fluid, one at a time, until each test tube is filled with several cubic centimeters of spinal fluid. Laboratories typically require four test tubes of spinal fluid from an adult patient, and three test tubes when the patient is a child, for example. Unfortunately, spinal fluid is often lost as the test tubes are switched. The physician must place the first full test tube carefully in a test tube holder, while picking up the empty second test tube and holding it under the dripping needle bore, etc. Once the second test tube fills, this procedure is repeated for the third and fourth test tubes, for example. Even if this is done as quickly as possible, significant amounts of spinal fluid can be lost during the test tube exchange. Since the spinal fluid is so difficult to obtain, it is desirable to the patient, the physician, and the hospital to expedite this process and lessen discomfort for the patient. It is of paramount importance that test tubes are not dropped, spilled, or contaminated.

Current test tubes used in most lumbar puncture trays have lids that are connected directly to the tubes and the design of these lids causes the lids to interfere with spinal fluid collection as the lid “floats” back into close proximity to the area via which the spinal fluid enters the test tube, making the process more difficult and increasing the risk of contamination.

One prior LP fluid collection device is provided in US 2005/0277848 (Graf), which provides a device for collecting spinal fluid during a lumbar puncture or other medical procedure including: a) an upper, generally cup-shaped portion including an open top, an upper cup side wall, an upper cup base plate at a lower end of the upper cup side wall, and a drain hole in the upper cup base plate; and b) a lower, generally cup-shaped portion including an open top, a lower cup side wall, and a lower cup base plate at a lower end of the lower cup side wall, the lower cup base plate including a plurality of generally circular openings; wherein the upper, generally cup-shaped portion is removably insertable in and rotatable in the lower, generally cup-shaped portion, and the upper, generally cup-shaped portion includes a rotation mechanism for rotating the upper, generally cup-shaped portion in the lower cup-shaped portion.

What is still needed in the art, however, is a simple and inexpensive sterile fluid handling device that may be used to efficiently hold and quickly fill the test tubes used in a LP procedure, without the test tubes being dropped, spilled, or contaminated.

### BRIEF SUMMARY OF THE INVENTION

In various exemplary embodiments, the present invention provides a holder for holding a set of LP test tubes together and allow spinal fluid collection to proceed (in test tube succession) without moving ones hands to and from sterile fields over and over. It holds the test tubes securely, so that there is much less chance of dropping, spillage, and/or contamination. By obviating the need for moving back and forth between the sterile field of the tray and the patient, it speeds up the process, and creates much less likelihood of spinal fluid spillage and accidental loss. It makes the test tubes much less likely to fall and spill, and helps lessen the opportunity to lose sterility in the LP procedure. An optional section of the holder can be used to hold the inner stylet of the LP needle while fluid is being collected, which lessens the clinician's likelihood of being accidentally stuck with a sharp object, which would both expose the clinician to the patient's bodily fluids and contaminate the field. This also keeps the stylet in close proximity to the clinician's hand, such that he or she can quickly replace the stylet in the needle once spinal fluid collection is complete.

In one exemplary embodiment, the present invention provides a sterile fluid handling device, comprising: a plate structure; one of a plurality of holes, a plurality of raised wells, and a plurality of sunken wells disposed in the plate structure, wherein each of the holes or wells is sized and configured to selectively receive and retain a corresponding fluid collection tube; and a gripping portion by which the plate structure is grasped by a user and moved while the fluid collection tubes are retained in the holes or wells. The device further comprising an elongate recess manufactured into the plate structure, wherein the elongate recess is sized and configured to receive an inner stylet of a fluid collection needle while the procedure is being done. The device further comprising a labeling portion on which a label is affixed to the plate structure. The holes or wells are arranged in a substantially linear pattern spanning a dimension of the plate structure. Alternatively, the holes or wells are arranged in a substantially curved pattern spanning a dimension of the plate structure. Each of the fluid collection tubes comprises an independent or coupled cap. Fluid collection tube identifiers may be printed or engraved on the plate structure adjacent to each hole or well and cap. A plurality of cap holders are preferably disposed on the surface of the plate structure for securely holding the caps from the collection tubes when not in use. These cap holders may include either raised or recessed concentric structures onto or into which the caps are pressed. Thus, the holder allows the caps to be secured in close proximity to, but away from, the associated collection tubes, such that these caps are not lost or contaminated, but also do not interfere with the procedure, and are readily accessible when they are needed to close the collection tubes.

In another exemplary embodiment, the present invention provides a sterile fluid handling device, comprising: a plate structure; one of a plurality of semicircular recesses disposed in the plate structure, wherein each of the recesses is sized and configured to selectively receive and retain a corresponding fluid collection tube; and a gripping portion by which the plate structure is grasped by a user and moved while the fluid collection tubes are retained in the recesses. The device further comprising an elongate recess manufactured into the plate structure, wherein the elongate recess is sized and configured to receive an inner stylet of a fluid collection needle while the procedure is being done. The device further comprising a labeling portion on which a label is affixed to the plate structure. The recesses are arranged in a substantially linear pattern along an edge of the plate structure. Alternatively, the recesses are arranged in a substantially linear pattern along a surface of the plate structure. Each of the fluid collection tubes comprises an independent or coupled cap. Fluid collection tube identifiers may be printed or engraved on the plate structure adjacent to each hole or well and cap. Again, a plurality of cap holders are preferably disposed on the surface of the plate structure for securely holding the caps from the collection tubes when not in use. These cap holders may include either raised or recessed concentric structures onto or into which the caps are pressed. Thus, the holder allows the caps to be secured in close proximity to, but away from, the associated collection tubes, such that these caps are not lost or contaminated, but also do not interfere with the procedure, and are readily accessible when they are needed to close the collection tubes.

In a further exemplary embodiment, the present invention provides a sterile fluid handling method, comprising: providing a sterile fluid handling device comprising a plate structure defining a plurality of holes, wells, or recesses; coupling a plurality of fluid collection tubes to the plate structure using the plurality of holes, wells, or recesses; and successively filling the plurality of fluid collection tubes with a bodily fluid by successively aligning each of the plurality of fluid collection tubes with a fluid collection needle bore and manually translating the plate structure of the sterile fluid handling device such that the fluid collection tubes move in unison.

### BRIEF DESCRIPTION OF THE DRAWINGS

The present invention is illustrated and described herein with reference to the various drawings, in which like reference numbers are used to denote like device components/method steps, as appropriate, and in which:

FIG. 1 is a schematic diagram illustrating one exemplary embodiment of the sterile fluid handling device of the present invention;

FIG. 2 is a schematic diagram illustrating another exemplary embodiment of the sterile fluid handling device of the present invention;

FIG. 3 is a schematic diagram illustrating one exemplary embodiment of a test tube used with the sterile fluid handling device of the present invention; and

FIG. 4 is a schematic diagram illustrating a further exemplary embodiment of the sterile fluid handling device of the present invention.

### DETAILED DESCRIPTION OF THE INVENTION

Again, in various exemplary embodiments, the present invention provides a holder for holding a set of LP test tubes together and allow spinal fluid collection to proceed (in test tube succession) without moving ones hands to and from sterile fields over and over. It holds the test tubes securely, so that there is much less chance of dropping, spillage, and/or contamination. By obviating the need for moving back and forth between the sterile field of the tray and the patient, it speeds up the process, and creates much less likelihood of spinal fluid spillage and accidental loss. It makes the test tubes much less likely to fall and spill, and helps lessen the opportunity to lose sterility in the LP procedure. An optional section of the holder can be used to hold the inner stylet of the LP needle while fluid is being collected, which lessens the clinician's likelihood of being accidentally stuck with a sharp object, which would both expose the clinician to the patient's bodily fluids and contaminate the field. This also keeps the stylet in close proximity to the clinician's hand, such that he or she can quickly replace the stylet in the needle once spinal fluid collection is complete.

In various exemplary embodiments, the present invention also provides a better-designed (or better-used) test tube, with a top that screws on and off completely or has a connection mechanism coupling it to the test tube that prevents it from “floating” back to the opening of the test tube. Although these test tubes are not the primary focus of the present invention, in the removable-cap-configuration, the holder of the present invention allows the caps to be secured in close proximity to, but away from, the associated test tubes, such that these caps are not lost or contaminated, but also do not interfere with the procedure, and are readily accessible when they are needed to close the test tubes.

Together, these two devices, the collection tube and the holder, may be inserted into a standard lumbar puncture tray by a manufacturer, or may be available as a separate package. No similar sterile product is known to exist for assisting in LP spinal fluid collection.

The present invention provides a new sterile device that is used to make the LP procedure more likely to successfully collect spinal fluid without wasting fluid or dropping a test tube, while ensuring a safer sterile field, since the clinician moves to and from the field a lower number of times during spinal fluid collection. The new test tube and cap mechanism allows for easier collection of the spinal fluid.

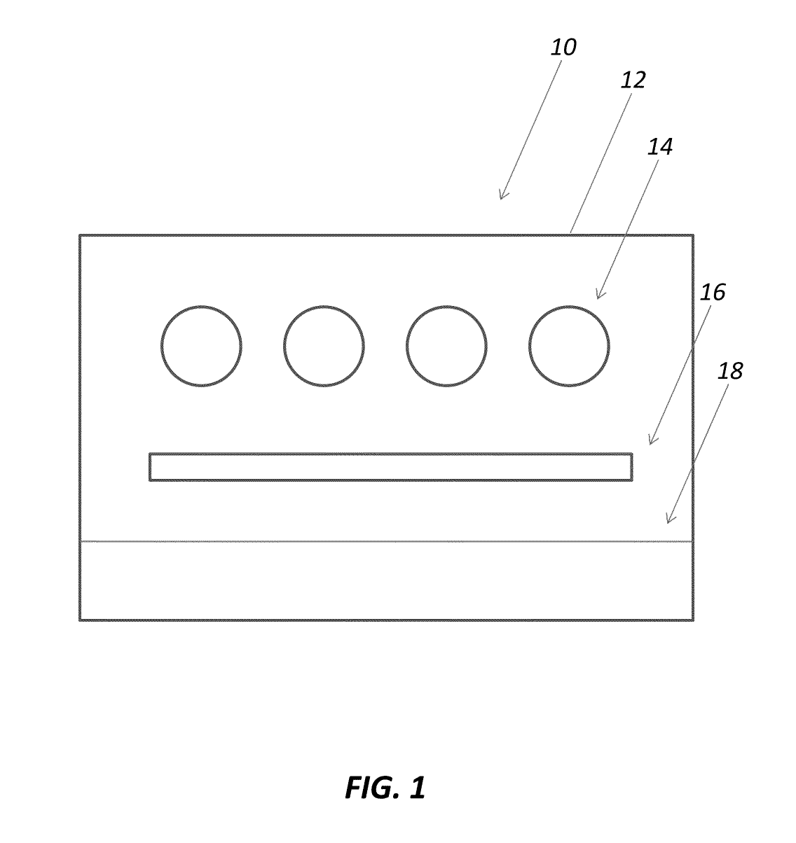

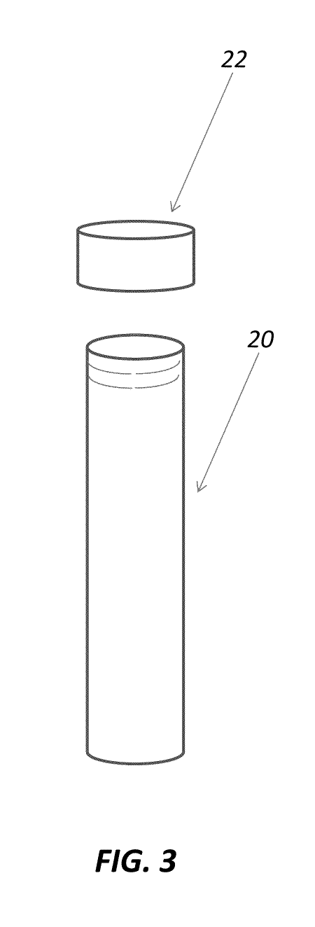

Referring now specifically to FIG. 1, in one exemplary embodiment of the present invention, the sterile fluid handling device 10 includes a plate structure 12 made of a plastic material or the like with four circular holes or raised/sunken wells 14 that the four spinal fluid collection tubes 20 (FIG. 3) are selectively inserted into. The plate structure 12 includes an elongate depression or well 16 sized and configured to selectively receive and retain an inner stylet of a LP needle. The plate structure 12 also includes an area 18 by which the plate structure 12 is grasped by the clinician and for a patient identification sticker to be attached. There may also be the numbers 1-4 imprinted or engraved on the device 10, which would help the clinician know which tubes go in which slots. Of course, any other numbers of holes or wells 14 may also be utilized. As is described in greater detail herein below, a plurality of cap holders are preferably disposed on the surface of the plate structure 12 for securely holding the caps 22 (FIG. 3) from the collection tubes 20 when not in use. These cap holders may include either raised or recessed concentric structures onto or into which the caps 22 are pressed. Thus, the holder 10 allows the caps 22 to be secured in close proximity to, but away from, the associated collection tubes 20, such that these caps 22 are not lost or contaminated, but also do not interfere with the procedure, and are readily accessible when they are needed to close the collection tubes 20.

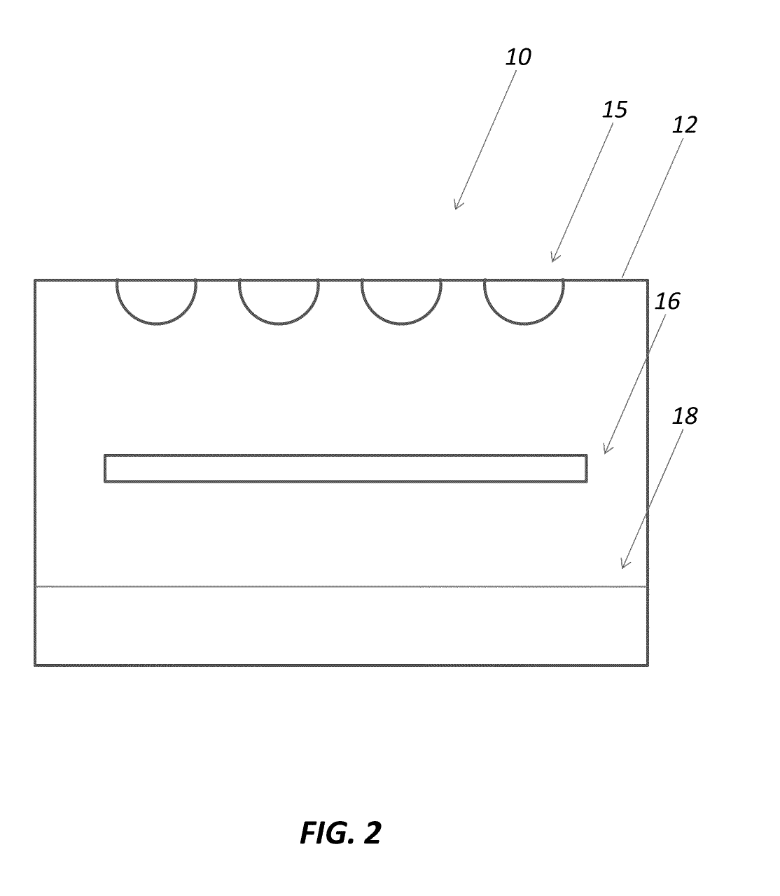

Referring now specifically to FIG. 2, in another exemplary embodiment of the present invention, the sterile fluid handling device 10 includes a plate structure 12 made of a plastic material or the like with four semicircular cutouts or recessed wells 15 that the four spinal fluid collection tubes 20 (FIG. 3) are selectively inserted into. The plate structure 12 includes an elongate depression or well 16 sized and configured to selectively receive and retain an inner stylet of a LP needle. The plate structure 12 also includes an area 18 by which the plate structure 12 is grasped by the clinician and for a patient identification sticker to be attached. There may also be the numbers 1-4 imprinted or engraved on the device 10, which would help the clinician know which tubes go in which slots. Of course, any other numbers of holes or wells 14 may also be utilized. Again, as is described in greater detail herein below, a plurality of cap holders are preferably disposed on the surface of the plate structure 12 for securely holding the caps 22 (FIG. 3) from the collection tubes 20 when not in use. These cap holders may include either raised or recessed concentric structures onto or into which the caps 22 are pressed. Thus, the holder 10 allows the caps 22 to be secured in close proximity to, but away from, the associated collection tubes 20, such that these caps 22 are not lost or contaminated, but also do not interfere with the procedure, and are readily accessible when they are needed to close the collection tubes 20.

Referring now specifically to FIG. 3, the tubes 20 are very similar to the current tubes in size, volume, and most other characteristics, except that the current mechanism for attaching the cap 22 to the tube 20 is abandoned and four separate detached caps 22 are used after fluid collection, or the mechanism by which the caps 22 are attached to the tubes 20 prevents the caps 22 from “floating” up into the field of use. This can be achieved with a variety of different materials and methods, namely a thinner and less durable material that allows the caps 22 to simply fall further away from the tubes 20 while still being attached. These new tubes 20 may or may not have numbers imprinted or engraved on them in the molding process. Some clinicians order a variety of different tests on the fluid and numbering can be important. The clinician may also have the ability to write specific numbers on the caps 22 of the tubes 20. The caps 22 may have a number imprinted or engraved directly on them (1-4) or the clinician or nurse may write the tube number directly on the cap 22 or use another method of identifying which tube 20 is which.

All of the tube holder embodiments of the present invention perform the same task. The goal is to help the clinician more efficiently and rapidly be able to collect spinal fluid. The current procedure is often cumbersome and inefficient. The use of this device allows the clinician to have all the needed tubes located in one place and allow them to rapidly collect the spinal fluid without having to go back and forth to and from the sterile LP tray/kit.

The new tube concept works together with the holder by having the tubes placed and secured in it. Regardless of the arrangement of the new cap—either separate or attached—the caps will still screw onto the tubes after the spinal fluid is collected so that they may be transported to the lab for analysis.

In use, a clinician would secure the tubes that he/she needs in the order needed, then inserts the LP needle into the appropriate spinal space, removes and stores the inner stylet, and when fluid return begins, uses the present invention to hold each collection tube below the LP needle bore and keep the tubes together to quickly move between tubes, keeping them secure and upright and much less likely to fall from the clinician's hands due to slippery bodily fluids. After completing the procedure, the clinician will attach the caps onto the tubes and they are ready for transport to the lab for analysis.

The present invention may be used in any field where multiple tubes for sterile body fluid collection are necessary, such as diagnostic paracentesis, joint fluid sampling, suprabupic bladder aspiration, thoracentesis, and direct brain/CNS fluid sampling in neonates.

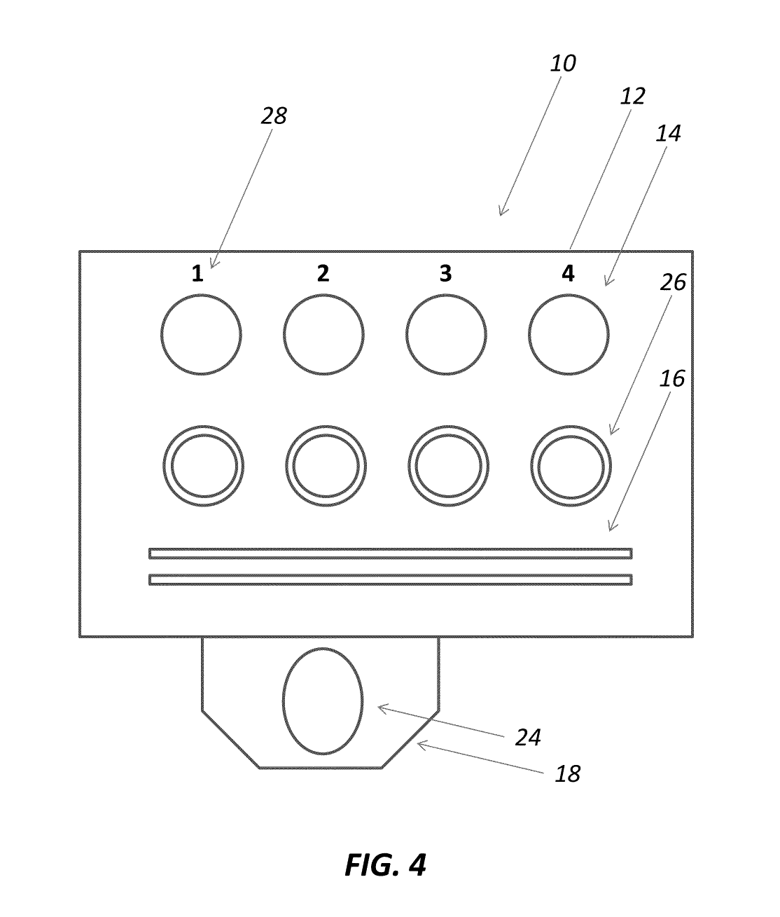

Referring now specifically to FIG. 4, in a further exemplary embodiment of the present invention, the sterile fluid handling device 10 includes a plate structure 12 made of a plastic material or the like (such as ABS, PC, PS, PE, PP, or the like) with four circular holes or raised/sunken wells 14 that the four spinal fluid collection tubes 20 (FIG. 3) are selectively inserted through or into such that they are secured. Printed or engraved numbers 28 are provided next to each of the holes or wells 14, such that each of the fluid collection tubes 20 can be readily identified at all times. The plate structure 12 includes an elongate depression or well 16 sized and configured to selectively receive and retain an inner stylet of a LP needle. In one exemplary embodiment, the elongate well 16 may include a pair of parallel raised structures in between which the inner stylet of the LP needle is disposed and/or secured, such that it is coupled to the plate structure 12 when not in use. The plate structure 12 also includes an area 18 by which the plate structure 12 is grasped by the clinician. As is illustrated, this area 18 may be a tab protruding from the plate structure, and may include a recessed thumb area 24 or the like. Again, there may also be printed or engraved numbers 28 on the device 10, which would help the clinician know which tubes go in which slots. Of course any other numbers of holes or wells 14 may also be utilized. Finally, in this exemplary embodiment, a plurality of cap holders or holes 26 are disposed on the surface of the plate structure 12 for securely holding the caps 22 (FIG. 3) from the collection tubes 20 when not in use. These cap holders 26 may include either raised or recessed concentric structures onto or into which the caps 22 are pressed. It will be readily apparent to those of ordinary skill in the art that other such structures could also be used.

Although the present invention is illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

What is claimed is:

1. A sterile fluid handling device, comprising:
a plate structure;
one of a plurality of holes, a plurality of raised wells, and a plurality of sunken wells disposed in the plate structure, wherein each of the holes or wells is sized and configured to selectively receive and retain a corresponding fluid collection tube; and
a gripping portion by which the plate structure is grasped by a user and moved while the fluid collection tubes are retained in the holes or wells.

2. The device of claim 1, further comprising an elongate channel manufactured into or onto the plate structure, wherein the elongate channel is sized and configured to receive an inner stylet of a fluid collection needle.

3. The device of claim 1, further comprising a labeling portion on which a label is affixed to the plate structure.

4. The device of claim 1, wherein the holes or wells are arranged in a substantially linear pattern spanning a dimension of the plate structure.

5. The device of claim 1, wherein the holes or wells are arranged in a substantially curved pattern spanning a dimension of the plate structure.

6. The device of claim 1, wherein each of the fluid collection tubes comprises an independent or coupled cap.

7. The device of claim 1, further comprising a plurality of raised or recessed concentric cap holding structures manufactured onto or into the plate structure.

8. A sterile fluid handling device, comprising:
a plate structure;
one of a plurality of semicircular recesses disposed in the plate structure, wherein each of the recesses is sized and configured to selectively receive and retain a corresponding fluid collection tube; and
a gripping portion by which the plate structure is grasped by a user and moved while the fluid collection tubes are retained in the recesses.

9. The device of claim 8, further comprising an elongate channel manufactured into or onto the plate structure, wherein the elongate channel is sized and configured to receive an inner stylet of a fluid collection needle.

10. The device of claim 8, further comprising a labeling portion on which a label is affixed to the plate structure.

11. The device of claim 8, wherein the recesses are arranged in a substantially linear pattern along an edge of the plate structure.

12. The device of claim 8, wherein the recesses are arranged in a substantially linear pattern along a surface of the plate structure.

13. The device of claim 8, wherein each of the fluid collection tubes comprises an independent or coupled cap.

14. The device of claim 8, further comprising a plurality of raised or recessed concentric cap holding structures manufactured onto or into the plate structure.

15. A sterile fluid handling method, comprising:
providing a sterile fluid handling device comprising a plate structure defining a plurality of holes, wells, or recesses;
coupling a plurality of fluid collection tubes to the plate structure using the plurality of holes, wells, or recesses; and
successively filling the plurality of fluid collection tubes with a bodily fluid by successively aligning each of the plurality of fluid collection tubes with a fluid collection needle bore and manually translating the plate structure of the sterile fluid handling device such that the fluid collection tubes move in unison.
